(19) United States
(12) Patent Application Publication
(10) Pub. No.: US 2026/0297559 A1
(43) Pub. Date: Oct. 1, 2026

(54) Biological Material Purification Method, Magnetic Bead, Magnetic Bead Dispersion Liquid, And Biological Material Purification Kit

(71) Applicant: SEIKO EPSON CORPORATION, Tokyo (JP)

(72) Inventor: Miki UCHIDA, Matsumoto (JP)

(21) Appl. No.: 19/629,200

(22) Filed: Mar. 26, 2026

(30) Foreign Application Priority Data
Mar. 27, 2025 (JP) 2025-053935

(51) Int. Cl. C12N 15/10

(52) U.S. Cl. CPC C12N 15/1013

(57) ABSTRACT

A biological material purification method includes: an adsorption step of adsorbing, in a mixed liquid obtained by mixing a magnetic bead and a biological material, the biological material to the magnetic bead; a separation step of separating, from the mixed liquid, the magnetic bead to which the biological material is adsorbed; and a desorption step of desorbing the biological material from the magnetic bead. The magnetic bead includes a magnetic metal particle, and a coating layer containing a cationic group. The following formulas (1), (2), and (3) are satisfied:

$$pH ( a ) \leq 7. \leq pH ( d ) \qquad ( 1 )$$

$$2. \leq pH ( d ) - pH ( a ) \qquad ( 2 )$$

$$C ( a ) \leq 2. [ M ] , \qquad ( 3 )$$

where pH(a) represents a pH of a liquid in the adsorption step, pH(d) represents a pH of a liquid in the desorption step, and C(a) represents a salt concentration of the liquid in the adsorption step.

The present application is based on, and claims priority from JP Application Serial Number 2025-053935, filed Mar. 27, 2025, the disclosure of which is hereby incorporated by reference herein in its entirety.

### BACKGROUND

### 1. Technical Field

The present disclosure relates to a biological material purification method, a magnetic bead, a magnetic bead dispersion liquid, and a biological material purification kit.

### 2. Related Art

JP-A-2001-352979 discloses a nucleic acid collection method including a step of bringing a cationic solid phase carrier into contact with a sample containing nucleic acids to capture the nucleic acids by the cationic solid phase carrier, a step of separating the cationic solid phase carrier capturing the nucleic acids from the sample, and a step of treating the separated cationic solid phase carrier with an anionic substance and separating the captured nucleic acid from the cationic solid phase carrier.

According to such a nucleic acid collection method, nucleic acids in a sample can be efficiently and simply collected with high purity.

JP-A-2001-352979 is an example of the related art.

However, in the nucleic acid collection method described in JP-A-2001-352979, the selectivity of the nucleic acid is not sufficient in the step of capturing the nucleic acid. In addition, the separability of the nucleic acid is low in the step of separating the captured nucleic acid. Therefore, in the nucleic acid collection method described in JP-A-2001-352979, there is a problem in that it is difficult to purify a high-purity nucleic acid at a high nucleic acid recovery rate (%).

### SUMMARY

A biological material purification method according to an application example of the present disclosure includes: an adsorption step of adsorbing, in a mixed liquid obtained by mixing a magnetic bead and a biological material, the biological material to the magnetic beads; a separation step of separating, from the mixed liquid, the magnetic bead to which the biological material is adsorbed; and a desorption step of desorbing the biological material from the magnetic bead. The magnetic bead includes a magnetic metal particle, and a coating layer covering a surface of the magnetic metal particle and containing a cationic group. The following formulas (1), (2), and (3) are satisfied:

$$pH ( a ) \leq 7. \leq pH ( d ) \qquad ( 1 )$$

$$2. \leq pH ( d ) - pH ( a ) \qquad ( 2 )$$

$$C ( a ) \leq 2. [ M ] , \qquad ( 3 )$$

in which pH(a) represents a pH of a liquid in which the magnetic bead is present in the adsorption step, pH(d) represents a pH of a liquid in which the magnetic bead is present in the desorption step, and C(a) represents a salt concentration of the liquid in which the magnetic bead is present in the adsorption step.

A magnetic bead according to an application example of the present disclosure is the magnetic bead used for the biological material purification method according to the application example of the present disclosure. The magnetic bead includes: the magnetic metal particle; and the coating layer configured to cover a surface of the magnetic metal particle. The coating layer includes a first layer containing at least one of an inorganic oxide and gold, and a second layer provided at the first layer and containing the cationic group.

A magnetic bead dispersion liquid according to an application example of the present disclosure contains: the magnetic bead according to the application example of the present disclosure; and a dispersion medium in which the magnetic bead is dispersed.

A biological material purification kit according to an application example of the present disclosure includes: the magnetic bead dispersion liquid according to the application example of the present disclosure.

### BRIEF DESCRIPTION OF THE DRAWINGS

FIG. 1 is a flowchart illustrating a configuration of a biological material purification method according to an embodiment.

FIG. 2 is a schematic diagram illustrating the biological material purification method illustrated in FIG. 1.

FIG. 3 is a schematic diagram illustrating the biological material purification method illustrated in FIG. 1.

FIG. 4 is a schematic diagram illustrating the biological material purification method illustrated in FIG. 1.

FIG. 5 is a cross-sectional view illustrating a magnetic bead according to the embodiment.

FIG. 6 is a partially enlarged view of the magnetic bead illustrated in FIG. 5.

FIG. 7 is a cross-sectional view schematically illustrating a biological material purification kit according to the embodiment.

FIG. 8 is Table 1 illustrating conditions for nucleic acid purification using magnetic beads in Examples and evaluation results of the nucleic acid purification.

FIG. 9 is Table 2 illustrating conditions for nucleic acid purification using magnetic beads in Examples and evaluation results of the nucleic acid purification.

FIG. 10 is Table 3 illustrating conditions for nucleic acid purification using magnetic beads in Examples and evaluation results of the nucleic acid purification.

FIG. 11 is Table 4 illustrating conditions for the nucleic acid purification using magnetic beads in Examples and Comparative Examples, and evaluation results of the nucleic acid purification.

### DESCRIPTION OF EMBODIMENTS

Hereinafter, preferred embodiments of a biological material purification method, a magnetic bead, a magnetic bead dispersion liquid, and a biological material purification kit according to the present disclosure will be described in detail with reference to the accompanying drawings.

The magnetic bead according to the embodiment is a particle group that adsorbs a biological material and is used for magnetic separation. Magnetic separation is a technique of applying an external magnetic field to a container containing a solid phase containing magnetic beads and a liquid phase containing a solvent or the like to magnetically adsorb the solid phase and thus separating the solid phase from the liquid phase.

Examples of the biological material include a nucleic acid such as DNA, RNA, and hybrids thereof. In addition, the biological material may be a biological component other than a nucleic acid, including an anion group such as a phosphate group. The nucleic acid or the biological component may be present in a state of being contained in, for example, a biological sample such as a cell or a biological tissue, a virus, or a bacterium. In the biological material purification method, such a biological material is purified through, for example, the steps of adsorption, separation, washing, and desorption.

### 1. Biological Material Purification Method

Hereinafter, an example of the biological material purification method using magnetic separation will be described. In the following description, a case where the biological material is a nucleic acid will be described as an example.

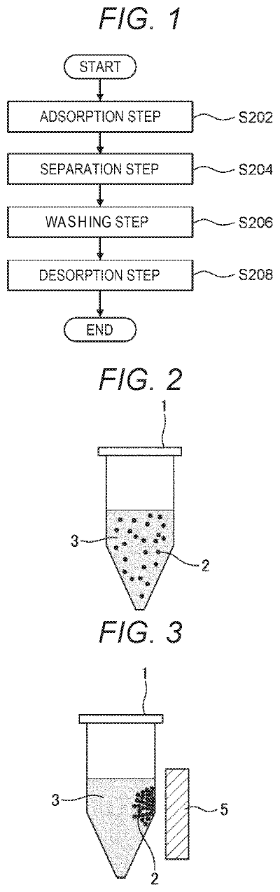

FIG. 1 is a flowchart illustrating a configuration of the biological material purification method according to the embodiment. FIGS. 2 to 4 are schematic diagrams illustrating the biological material purification method illustrated in FIG. 1.

### 1.1. Overview

The biological material purification method illustrated in FIG. 1 includes an adsorption step S202, a separation step S204, a washing step S206, and a desorption step S208.

In the adsorption step S202, the magnetic beads 2, a sample containing a nucleic acid (biological material) (not illustrated), and a nucleic acid adsorption liquid (liquid 3) are mixed in a container 1 illustrated in FIG. 2 to prepare a mixed liquid. Then, the nucleic acid is adsorbed to the magnetic bead 2 in the mixed liquid.

In the separation step S204, the magnetic beads 2 to which the nucleic acid is adsorbed are separated from the mixed liquid in the container 1.

In the washing step S206, the magnetic beads 2 to which the nucleic acid is adsorbed are cleaned in the container 1. In the present disclosure, the washing step S206 is not essential and may be omitted.

In the desorption step S208, the nucleic acid is desorbed from the magnetic beads 2 to which the nucleic acid is adsorbed to a nucleic acid desorption liquid (liquid 3) in the container 1.

The magnetic bead 2 includes a magnetic metal particle 22 and a coating layer 24. The coating layer 24 covers a surface of the magnetic metal particle 22 and contains a cationic group.

In the present embodiment, the pH of the liquid 3 in which the magnetic beads 2 are present in the adsorption step S202 is denoted by pH(a), and the pH of the liquid 3 in which the magnetic beads 2 are present in the desorption step S208 is denoted by pH(d). Further, a salt concentration of the liquid 3 in which the magnetic beads 2 are present in the adsorption step S202 is denoted by C(a). At this time, the embodiment satisfies the following formulas (1), (2), and (3).

$$pH ( a ) \leq 7. \leq pH ( d ) \qquad ( 1 )$$

$$2. \leq pH ( d ) - pH ( a ) \qquad ( 2 )$$

$$C ( a ) \leq 2. [ M ] \qquad ( 3 )$$

According to such a configuration, the nucleic acid can be efficiently adsorbed to the magnetic bead 2 by an ionic interaction between the magnetic bead 2 containing a cationic group and the nucleic acid containing a phosphate group. In addition, adsorption of contaminants can be prevented while achieving efficient adsorption and desorption of nucleic acids, by optimizing the pH of the liquids in the adsorption step S202 and the desorption step S208 and optimizing the salt concentration of the liquid in the adsorption step S202. As a result, it is possible to provide a biological material purification method capable of purifying a high-purity nucleic acid in a high recovery rate (%).

### 1.2. Adsorption Step

In the adsorption step S202, a sample containing the magnetic beads 2, the nucleic acid adsorption liquid (liquid 3), and the nucleic acid is charged into the container 1 illustrated in FIG. 2. Then, substances accommodated in the container 1 are mixed. Accordingly, as illustrated in FIG. 2, the magnetic beads 2 are dispersed in the nucleic acid adsorption liquid in the container 1. As a result, the nucleic acid comes into contact with the magnetic beads 2, and the nucleic acid is captured by the magnetic beads 2 through the ionic interaction. The ionic interaction in the present specification refers to an attractive force acting between the two because the nucleic acid is negatively charged and the magnetic bead 2 is positively charged. The nucleic acid can be efficiently adsorbed by utilizing such an ionic interaction. Accordingly, the recovery rate (%) of the nucleic acid in the adsorption step S202 can be increased.

When the ionic interaction is utilized, it is not necessary to use a substance that promotes adsorption of a nucleic acid to the magnetic bead 2, for example, a chaotropic substance. The chaotropic substance promotes the adsorption of the nucleic acid to the magnetic bead 2, but may be mixed into the nucleic acid collected in the desorption step S208. In addition, a chaotropic substance or the like can be prevented from mixing into the nucleic acid collected in the desorption step S208. Therefore, the washing step S206 in which cleaning needs to be performed a plurality of times using a plurality of cleaning liquids can be prevented from becoming complicated and taking too long. In the present embodiment, it is not necessary to use a chaotropic substance or the like, and therefore, such a concern can be eliminated.

The nucleic acid adsorption liquid in the adsorption step S202 is not particularly limited as long as it is a liquid for dispersing the magnetic beads 2, and examples thereof include water such as sterile water.

Any additive may be added to the nucleic acid adsorption liquid (liquid 3). Examples of the additive include a salt, a lysis agent, a buffer, an organic solvent, an acid, and a base.

Examples of the salt include sodium chloride, lithium chloride, ammonium sulfate, and trisodium citrate.

The lysis agent lyses and removes a cell membrane or an outer shell of a nucleus or the like of a biological sample to contribute to taking out the nucleic acid. Examples of the lysis agent include a triton surfactant such as Triton X-100, a nonionic surfactant such as a Tween surfactant such as Tween20, and an anionic surfactant such as sodium Dodecyl Sulfate (SDS). A concentration of the surfactant contained in the lysis agent is, for example, 0.5 mass % or more and 5 mass % or less.

Examples of the buffer include an acetate buffer, a phosphate buffer, a formic acid buffer, a citrate buffer, and a tartrate buffer.

Examples of the organic solvent include alcohols such as ethanol.

FIG. 5 is a cross-sectional view illustrating the magnetic bead 2 according to the embodiment.

The magnetic bead 2 illustrated in FIG. 5 contains the magnetic metal particle 22 and the coating layer 24. A metal powder having magnetism is used as the magnetic metal particle 22. The coating layer 24 contains a cationic group. The magnetic beads 2 will be described in detail below.

A zeta potential of the magnetic bead 2 in the adsorption step S202 is preferably a positive zeta potential of 20 [mV] or more. The magnetic beads 2 having such a zeta potential have sufficiently strong cationic properties. Therefore, in the magnetic beads 2 in the adsorption step S202, a stronger attractive force can be applied to the nucleic acid by the ionic interaction. Accordingly, the adsorption efficiency of the nucleic acid can be further increased. The zeta potential of the magnetic bead 2 can be controlled by adjusting an introduction amount of the cationic group and the pH of the liquid in which the magnetic beads 2 are present in the adsorption step S202.

### 1.3. Separation Step

In the separation step S204, an external magnetic field is applied to the mixed liquid containing the magnetic beads 2 to which the nucleic acid is adsorbed, and the magnetic beads 2 are magnetically attracted to an inner wall of the container 1. Accordingly, as illustrated in FIG. 3, the magnetic beads 2 are moved to the inner wall of the container 1 and immobilized. As a result, the magnetic beads 2 as a solid phase can be magnetically separated from the nucleic acid adsorption liquid (liquid 3) as a liquid phase. In the present specification, the treatment of magnetic separation is referred to as a “magnetic separation treatment”. In the biological material purification method according to the embodiment, the magnetic separation treatment is not essential, and instead of the magnetic separation treatment, a treatment of separating the magnetic beads 2 by a principle other than magnetic attraction may be performed.

Before the magnetic separation treatment is performed, the substances accommodated in the container 1 are stirred as necessary. Accordingly, a probability that the nucleic acid is captured by the magnetic beads 2 can be increased. In the stirring, for example, a vortex mixer, shaking by hand, or pipetting is used. This stirring is preferably performed in a state in which no external magnetic field is applied. Accordingly, the magnetic beads 2 are favorably dispersed in the nucleic acid adsorption liquid, and therefore, the adsorption efficiency can be increased.

For example, a magnet 5 disposed on a side of the container is used to apply the external magnetic field. The magnet 5 may be an electromagnet or a permanent magnet. When the external magnetic field acts on the magnetic beads 2, the magnetic beads 2 move toward the magnet 5.

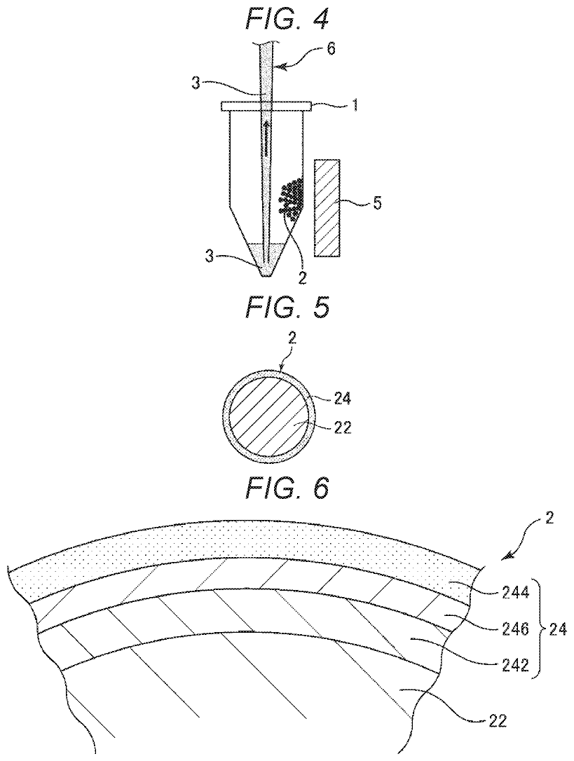

In a state in which the magnetic beads 2 are immobilized to the inner wall of the container 1 by performing the magnetic separation treatment, as illustrated in FIG. 4, the nucleic acid adsorption liquid (liquid 3) accumulated at a bottom of the container 1 is sucked and discharged by, for example, a pipette 6 or the like. In the present specification, the treatment of discharging the liquid 3 illustrated in FIG. 4 is referred to as a “liquid discharging treatment”. With the liquid discharging treatment, the magnetic beads 2 capturing the nucleic acid remain in the container 1.

After the liquid discharging treatment is performed, an acceleration may be applied to the container as necessary. Accordingly, the nucleic acid adsorption liquid adhering to the magnetic beads 2 can be shaken off, and thus the unseparated nucleic acid adsorption liquid can be reduced. The acceleration may be a centrifugal acceleration. A centrifugal separator may be used to apply the centrifugal acceleration.

### 1.4. Washing Step

In the washing step S206, the magnetic beads 2 to which the nucleic acid is adsorbed are cleaned. Cleaning refers to a treatment of transferring contaminants captured by the magnetic beads 2 to the cleaning liquid and removing the contaminants by bringing the magnetic beads 2, to which the nucleic acid is adsorbed, into contact with a cleaning liquid and then separating the magnetic beads 2 from the cleaning liquid again in order to remove the contaminants.

Specifically, after the cleaning liquid is charged into the container 1 subjected to the separation step S204, the magnetic separation treatment and the liquid discharging treatment described above are performed again. In this case, the liquid 3 in FIGS. 2 to 4 is a cleaning liquid.

In the magnetic separation treatment, first, the magnetic beads 2 and the cleaning liquid in the container 1 are stirred. Accordingly, the magnetic beads 2 are brought into contact with the cleaning liquid and cleaned. At this time, the application of the external magnetic field may be temporarily turned off. Accordingly, the magnetic beads 2 are re-dispersed in the cleaning liquid, and therefore, the cleaning efficiency can be increased. Thereafter, the external magnetic field is applied to immobilize the magnetic beads 2 to the inner wall of the container 1.

In the liquid discharging treatment, the cleaning liquid accumulated on the bottom of the container 1 is discharged in a state in which the magnetic beads 2 are immobilized to the inner wall of the container 1. Accordingly, the magnetic beads 2 are cleaned.

The supply and discharging of the cleaning liquid may be repeated twice or more in total. That is, cleaning may be performed a plurality of times. Accordingly, the contaminants can be accurately removed.

When the cleaning is performed a plurality of times, the components in the cleaning liquid may be different each time.

The cleaning liquid is not particularly limited as long as the cleaning liquid is a liquid that does not promote desorption of the nucleic acid and does not promote binding of the contaminant to the magnetic beads 2, and examples thereof include organic solvents such as ethanol, isopropyl alcohol, and acetone, aqueous solutions thereof, and low-salt-concentration aqueous solutions, in addition to water.

Various additives may be added to the cleaning liquid. Examples of the additives include buffers such as an acetic acid buffer and a phosphoric acid buffer, salts such as sodium chloride, and surfactants.

The washing step S206 may be performed as necessary and may be omitted when cleaning is not necessary.

### 1.5. Desorption Step

In the desorption step S208, the nucleic acid captured by the magnetic beads 2 is eluted into the nucleic acid desorption liquid. Desorption is a treatment of transferring the nucleic acid to the nucleic acid desorption liquid by bringing the magnetic bead 2 to which the nucleic acid is adsorbed into contact with the nucleic acid desorption liquid.

Specifically, after the nucleic acid desorption liquid is charged into the container 1 containing the magnetic beads 2 to which the nucleic acid is adsorbed, the magnetic separation treatment and the liquid discharging treatment described above are performed again. In this case, the liquid 3 in FIGS. 2 to 4 is a nucleic acid desorption liquid.

In the magnetic separation treatment, first, the magnetic beads 2 and the nucleic acid desorption liquid in the container 1 are stirred. Accordingly, the nucleic acid desorption liquid comes into contact with the magnetic beads 2, and the nucleic acid is eluted into the nucleic acid desorption liquid. At this time, the application of the external magnetic field may be temporarily turned off. Accordingly, the magnetic beads 2 are re-dispersed in the nucleic acid desorption liquid, and therefore, the desorption efficiency can be increased. Thereafter, the external magnetic field is applied to immobilize the magnetic beads 2 to the inner wall of the container 1.

In the liquid discharging treatment, the nucleic acid desorption liquid accumulated at the bottom of the container 1 is discharged in a state in which the magnetic beads 2 are fixed to the inner wall of the container 1. Accordingly, the nucleic acid desorption liquid containing nucleic acid can be collected.

The nucleic acid desorption liquid is not particularly limited as long as it is a liquid that promotes desorption of the nucleic acid captured by the magnetic beads 2, and examples thereof include water such as sterile water and pure water.

Any additive may be added to the nucleic acid desorption liquid. Examples of the additive include a salt, a buffer, a surfactant, an organic solvent, an acid, and a base.

Examples of the salt include sodium chloride, lithium chloride, potassium carbonate, and trisodium citrate.

The zeta potential of the magnetic bead 2 in the desorption step S208 is preferably a zeta potential of 0 [mV] or less. The magnetic beads 2 having such a zeta potential have sufficiently weak cationic properties. Therefore, in the magnetic beads 2 in the desorption step S208, the ionic interaction is weakened, and the desorption efficiency of the nucleic acid can be increased.

### 1.6. Conditions in Adsorption Step and Desorption Step

Next, the conditions in the adsorption step S202 and the desorption step S208 will be described.

### 1.6.1. pH and Salt Concentration of Solvent

The pH of the nucleic acid adsorption liquid in the adsorption step S202 (the liquid 3 in which the magnetic beads 2 are present in the adsorption step S202) is denoted by pH(a). The pH of the nucleic acid desorption liquid in the desorption step S208 (the liquid 3 in which the magnetic beads 2 are present in the desorption step S208) is denoted by pH(d). At this time, the biological material purification method according to the embodiment satisfies the following formulas (1) and (2).

$$pH ( a ) \leq 7. \leq pH ( d ) \qquad ( 1 )$$

$$2. \leq pH ( d ) - pH ( a ) \qquad ( 2 )$$

When the above formulas (1) and (2) are satisfied, the pH(a) of the nucleic acid adsorption liquid in the adsorption step S202 is 7.0 or less, the pH of the nucleic acid desorption liquid in the desorption step S208 is 7.0 or more, and a difference between the pH(a) and the pH(d) is 2.0 or more.

In the adsorption step S202, the salt concentration of the nucleic acid adsorption liquid is denoted by C(a). At this time, the biological material purification method according to the present embodiment satisfies the following formula (3).

$$C ( a ) \leq 2. [ M ] \qquad ( 3 )$$

When the above formula (3) is satisfied, the salt concentration C(a) of the nucleic acid adsorption liquid in the adsorption step S202 is 2.0 [M] or less.

According to such a configuration, the adsorption efficiency of the nucleic acid to the magnetic beads 2 is increased in the adsorption step S202, and the desorption efficiency of the nucleic acid is increased in the desorption step S208. In the adsorption step S202, when the pH(a) and the C(a) are optimized, the surface charges of the magnetic beads 2 or ion exchange by the salts is controlled to a range suitable for adsorption, and the adsorption efficiency of the nucleic acid increases. Specifically, in the adsorption step S202, the cationic strength of the magnetic beads 2 can be increased, and the ionic interaction is strengthened. Therefore, the adsorption efficiency of the nucleic acid can be increased.

In the desorption step S208, when the pH(d) is optimized, the surface charges of the magnetic beads 2 are controlled to a range suitable for desorption, and the desorption efficiency of the nucleic acid increases. Specifically, in the desorption step S208, the cationic property of the magnetic beads 2 can be weakened, and the ionic interaction is weakened, and therefore, the desorption efficiency of the nucleic acid can be increased. Accordingly, the recovery rate (%) of the nucleic acid finally collected from the nucleic acid desorption liquid can be increased.

When the pH(a) and the C(a) are optimized in the adsorption step S202, the adsorption efficiency of contaminants to the magnetic beads 2 decreases. Specifically, the pH(a) of the nucleic acid adsorption liquid is likely to be equal to or lower than an isoelectric point of the contaminants, and adsorption of contaminants can be prevented. In addition, the salt concentration C(a) of the nucleic acid adsorption liquid prevents a decrease in the adsorption of the nucleic acid while preventing the adsorption of the contaminants. Accordingly, the purity of the nucleic acid finally collected from the nucleic acid desorption liquid can be increased. Examples of the contaminants include proteins.

When the pH(a) and the pH(d) do not satisfy the above formula (1) or (2), or when the C(a) does not satisfy the above formula (3), the surface charges of the magnetic beads 2 or the ion exchange by the salts cannot be favorably controlled, the adsorption efficiency and the desorption efficiency of the nucleic acid cannot be sufficiently increased, or the adsorption efficiency of contaminants cannot be sufficiently reduced.

In the adsorption step S202, the pH(a) is measured for a supernatant obtained by precipitating only the magnetic beads 2 in a state in which the sample containing nucleic acids, the magnetic beads 2, and the nucleic acid adsorption liquid (liquid 3) are present.

Similarly, in the desorption step S208, the pH(d) is measured for a supernatant obtained by precipitating only the magnetic beads 2 in a state in which the magnetic beads 2 and the nucleic acid desorption liquid (liquid 3) having undergone the previous step are present.

The pH(a) is preferably 2.0 or more and 6.5 or less, and more preferably 3.0 or more and 6.0 or less. When the pH(a) is lower than the lower limit, the nucleic acid or the magnetic beads 2 may be denatured under the influence of pH(a).

The pH(d) is preferably 7.2 or more and 12.0 or less, and more preferably 7.4 or more and 11.0 or less. When the pH(d) is more than the above upper limit, the nucleic acid or the magnetic beads 2 may be denatured under the influence of pH(d).

The pH(d)−pH(a) is preferably 2.5 or more and 8.5 or less, and more preferably 3.0 or more and 8.0 or less. When the pH(d)−pH(a) is more than the above upper limit, the balance between the pH(a) and the pH(d) collapses, and it may be difficult to prevent the adsorption of contaminants while enhancing both the adsorption efficiency of the nucleic acid and the desorption efficiency of the nucleic acid.

### 1.6.2. Temperature of Solvent

The temperature of the nucleic acid adsorption liquid in the adsorption step S202 (the liquid 3 in which the magnetic beads 2 are present in the adsorption step S202) is denoted by T(a). The temperature of the nucleic acid desorption liquid in the desorption step S208 (the liquid 3 in which the magnetic beads 2 are present in the desorption step S208) is denoted by T(d). At this time, the biological material purification method according to the present embodiment preferably satisfies the following formulas (4), (5), and (6).

$$T ( a ) \leq T ( d ) \qquad ( 4 )$$

$$T ( a ) \leq 35 [ ° C . ] \qquad ( 5 )$$

$$20 [ ° C . ] \leq T ( d ) \qquad ( 6 )$$

When the above formulas (4), (5), and (6) are satisfied, the temperature T(a) of the nucleic acid adsorption liquid in the adsorption step S202 is equal to or lower than the temperature T(d) of the nucleic acid desorption liquid in the desorption step S208 and is 35[° C.] or lower. The temperature T(d) of the nucleic acid desorption liquid in the desorption step S208 is equal to or higher than the temperature T(a) of the nucleic acid adsorption liquid in the adsorption step S202, and is 20[° C.] or higher. Accordingly, the temperature T(a) of the nucleic acid adsorption liquid and T(d) of the nucleic acid desorption liquid are optimized, respectively, and the adsorption efficiency of the nucleic acid in the adsorption step S202 and the desorption efficiency of the nucleic acid in the desorption step S208 can be increased. Specifically, in the adsorption step S202, exchange of the phosphate group in the nucleic acid captured by the magnetic beads 2 with other anions is prevented, and thus, more nucleic acids can be captured. On the other hand, in the desorption step S208, the phosphate group in the nucleic acid captured by the magnetic bead 2 is promoted to be exchanged with other anions, and therefore, more nucleic acids can be desorbed.

T(a) is preferably 5° C. or higher and 32° C. or lower, and more preferably 15° C. or higher and 30° C. or lower. When T(a) is lower than the lower limit, the adsorption efficiency of the nucleic acid may decrease.

T(d) is preferably 22° C. or higher and 80° C. or lower, and more preferably 24° C. or higher and 75° C. or lower. When T(d) is higher than the above upper limit, the nucleic acid may be thermally denatured.

T(d)−T(a) may be 0° C. or higher, preferably 0° C. or higher and 60° C. or lower, and more preferably 0° C. or higher and 50° C. or lower. When T(d)−T(a) is more than the above upper limit, at least one of T(a) and T(d) may not be optimized.

### 2. Magnetic Beads

Next, the magnetic bead 2 (magnetic bead according to the embodiment) will be described.

The magnetic bead 2 illustrated in FIG. 5 contains the magnetic metal particle 22 and the coating layer 24. The coating layer 24 preferably covers the entire surface of the magnetic metal particle 22, but may have a discontinuous portion.

### 2.1. Magnetic Metal Particle

The magnetic metal particle 22 is a metal particle having magnetism.

Examples of a constituent material of the magnetic metal particle 22 include an Fe-based alloy, a Co-based alloy, and an Ni-based alloy. In particular, from the viewpoint of obtaining high saturation magnetization, the constituent material of the magnetic metal particle 22 is preferably an Fe-based alloy (alloy containing Fe as a main component).

The Fe-based alloy may be an alloy having a content of Fe of 50% or more in terms of atomic ratio, and the content of Fe is preferably 70% or more.

The Fe-based alloy contains Fe as a main component, and may contain one or two or more selected from the group including Co, Ni, Cr, Nb, Cu, Al, Mn, Mo, Si, Sn, B, C, P, Ti, and Zr, depending on target characteristics.

An example of the Fe-based alloy is an alloy having a content of Si of preferably 1.0 atomic % or more and 30.0 atomic % or less, more preferably 1.5 atomic % or more and 13.0 atomic % or less, and still more preferably 2.0 atomic % or more and 7.0 atomic % or less. Since such an alloy has a high magnetic permeability, the saturation magnetization tends to be high.

The Fe-based alloy may contain at least one of boron (B) having a content of 5.0 atomic % or more and 16.0 atomic% or less and carbon (C) having a content of 0.5 atomic % or more and 5.0 atomic % or less. These elements are elements that promote amorphization, and contribute to stably forming an amorphous structure or a nanocrystal structure in the magnetic metal particle 22.

Further, the Fe-based alloy may contain Cr (chromium) having a content of 1.0 atomic % or more and 8.0 atomic % or less. Accordingly, corrosion resistance of the magnetic metal particle 22 can be improved.

A content of impurities is preferably 1.0 atomic % or less in total. At this level, the above effect exhibited by the magnetic metal particle 22 is less likely to be impaired even when impurities are contained. The impurity in the present specification is an element that is unintentionally mixed in raw materials of the magnetic metal particle 22 or during the production of the magnetic metal particle 22.

A main metal structure forming the magnetic metal particle 22 may take various forms such as a crystal structure, an amorphous structure, and a nanocrystal structure. An amorphous structure refers to a non-crystal structure in which no crystal is present, and a nanocrystal refers to a structure mainly formed of fine crystals having a crystal grain size of 100 nm or less. The amorphous structure and the nanocrystal structure impart a high hardness to the magnetic metal particle 22. When the structure is the amorphous structure or the nanocrystal structure, the coercive force of the magnetic beads 2 has a particularly low value, which contributes to improvement in the redispersibility of the magnetic beads 2. A volume fraction of the magnetic metal particle 22 having an amorphous structure or a nanocrystal structure is preferably 40% or more, and more preferably 60% or more. The volume fraction is determined based on a result of crystal structure analysis using X-ray diffraction.

The metal structure of the magnetic metal particle 22 can also be identified by performing crystal structure analysis using an X-ray diffraction method on the magnetic metal particle 22. Alternatively, the metal structure can be specified by analyzing a structure observation image or a diffraction pattern obtained with a transmission electron microscope (TEM) from a cut-out sample. For example, in the case of the amorphous structure, a diffraction peak derived from a metal crystal of an α-Fe phase or the like is not observed in peak analysis in the X-ray diffraction method. In the case of the amorphous structure, a so-called halo pattern is formed in an electron diffraction pattern using TEM, and formation of a spot due to a crystal is not observed. The nanocrystal structure is formed of a crystal structure having a grain diameter of, for example, 100 nm or less, and can be checked from a TEM observation image.

The magnetic metal particle 22 preferably contains an Fe-based amorphous alloy in particular. The Fe-based amorphous alloy is an Fe-based alloy including an amorphous structure. Such a magnetic metal particle 22 has a high magnetic permeability and a low coercive force. Therefore, the magnetic beads 2 having good separability in magnetic separation and excellent redispersibility can be obtained by using such a magnetic metal particle 22.

### 2.2. Coating Layer

The coating layer 24 may be any layer that covers the magnetic metal particle 22 and contains cationic groups on the surface. Alternatively, the coating layer 24 has a multilayer structure, and an outermost layer preferably has a structure containing a cationic group. According to such a configuration, for example, a function of protecting the magnetic metal particle 22 can be imparted to the layer located inward than the outermost layer. Accordingly, it is possible to achieve the magnetic bead 2 having the above effect by the cationic group while preventing elution of metal ions or the like from the magnetic metal particle 22.

FIG. 6 is a partially enlarged view of the magnetic bead 2 illustrated in FIG. 5.

The coating layer 24 illustrated in FIG. 6 includes a first layer 242, a second layer 244, and a third layer 246. The first layer 242 contains at least one of an inorganic oxide and gold. The second layer 244 is an outermost layer of the coating layer 24 and contains cationic groups. The third layer 246 is provided between the first layer 242 and the second layer 244.

### 2.2.1. First Layer

The first layer 242 covers the surface of the magnetic metal particle 22 and contains at least one of an inorganic oxide and gold. Examples of the inorganic oxide include a silicon oxide, a magnesium oxide, a calcium oxide, an aluminum oxide, a titanium oxide, a zirconium oxide, a boron oxide, a yttrium oxide, and a molybdenum oxide, and a mixture of one or two or more thereof may be used. The first layer 242 containing such an inorganic oxide is porous, and therefore, a large contact area with the third layer 246 can be ensured. Accordingly, the adhesion between the two can be increased.

When the magnetic beads 2 are subjected to the above-described biological material purification method, the first layer 242 may be provided as necessary, and may be omitted. For example, when a large number of hydroxy groups are present on the surface of the magnetic metal particle 22, the magnetic metal particle 22 and the second layer 244 can easily adhere to each other without using the first layer 242. There is a tendency that a large number of hydroxy groups are present on the surface of the magnetic metal particle 22 produced by an atomizing method described later or the like.

The inorganic oxide is preferably a silicon oxide or a titanium oxide. The silicon oxide and the titanium oxide are chemically stable, and therefore, oxidation and corrosion of the magnetic metal particle 22 can be particularly reduced, and the corrosion resistance of the magnetic bead 2 can be particularly improved.

The silicon oxide is represented by a composition formula of SiOₓ (0<x≤2), and is preferably SiO₂. The silicon oxide may form a composite oxide or a composite with one or two or more selected from the group including Al, Ti, V, Nb, Cr, Mn, Sn, and Zr.

The titanium oxide is represented by a composition formula of TiOₓ (0<x≤2), and is preferably TiO₂. The titanium oxide may form a composite oxide or a composite with one or two or more selected from the group including Si, Al, V, Nb, Cr, Mn, Sn, and Zr.

The first layer 242 may contain a substance (impurity) other than the inorganic oxide within a range in which an effect thereof is not impaired, for example, at a ratio of 50 mass % or less of the inorganic oxide described above. When the silicon oxide is used as the inorganic oxide, examples of the impurity include C, N, and P.

A composition of the inorganic oxide can be checked by, for example, EDX analysis or Auger electron spectroscopy.

The thickness of the first layer 242 containing an inorganic oxide is preferably 10 nm or more and 200 nm or less, more preferably 15 nm or more and 100 nm or less, and still more preferably 20 nm or more and 100 nm or less. Accordingly, even when the magnetic beads 2 collide with each other or collide with the inner wall of the container 1 or the like, the first layer 242 can be prevented from being broken or peeled off. As a result, elution of iron ions and the like due to exposure of the magnetic metal particles 22 can be prevented. A decrease in the magnetization per unit volume of the magnetic beads 2 can be prevented, and a decrease in the movement speed of the magnetic beads 2 can be prevented.

Gold is a good base of the third layer 246 or the second layer 244. In addition, the gold has high corrosion resistance, and therefore, a function of preventing oxidation and corrosion of the magnetic metal particle 22 is high. Accordingly, it is possible to prevent a reduction in the magnetization of the magnetic metal particle 22 and prevent a decrease in the adsorption efficiency of the nucleic acid due to elution of iron ions or the like.

Gold may be present in a state of a simple substance, an alloy, or the like. When the first layer 242 contains gold, the first layer 242 may contain impurities other than gold.

The thickness of the gold-containing first layer 242 is preferably 0.5 nm or more and 50 nm or less, more preferably 1 nm or more and 30 nm or less, and still more preferably 2 nm or more and 20 nm or less. Accordingly, the corrosion resistance of the magnetic beads 2 can be particularly favorably ensured. A decrease in the magnetization per unit volume of the magnetic beads 2 can be prevented, and a decrease in the movement speed of the magnetic beads 2 can be prevented.

The first layer 242 may cover a surface of one magnetic metal particle 22, or may cover a plurality of magnetic metal particles 22 together. In this case, the same applies to the second layer 244 and the third layer 246.

The thickness of the first layer 242 can be measured from, for example, a cross-sectional observation image of the magnetic bead 2 observed with a transmission electron microscope or a scanning electron microscope. Specifically, the thickness of the first layer 242 can be calculated by acquiring a plurality of cross-sectional observation images of the first layer 242 and averaging measured values from image processing or the like. For example, the thickness of the first layer 242 is measured at five or more positions for one magnetic bead 2, an average value is determined, and then the average value is further averaged for ten or more magnetic beads 2.

### 2.2.2. Second Layer

The second layer 244 is an outermost layer provided at the first layer 242 and includes cationic groups.

A method of forming the second layer 244 is not particularly limited, and examples thereof include a method of forming a substance containing a cationic group on a base, and a method of causing a substance containing a cationic group to react with a base.

In the present specification, examples of the cationic group include a group that forms a cation by itself, and a group that does not form a cation by itself, but easily forms a cation by being bonded to a proton by changing the pH of the liquid in which the magnetic beads 2 are present. Examples of the former group include a quaternary ammonium group derived from a quaternary ammonium salt. Examples of the latter group include a primary amino group, a secondary amino group, a tertiary amino group, and an imino group. Examples of a group that can fall within both the former group and the latter group include groups derived from a cationic polymer.

The quaternary ammonium salt is a salt containing an ammonium cation with four substituents and a counter ion (anion).

Examples of the quaternary ammonium salt include trimethyl[3-(trimethoxysilyl)propyl]ammonium chloride, benzyltrimethylammonium chloride, dimethyloctadecyl[3-(trimethoxysilyl)propyl]ammonium chloride, dodecyldimethyl γ-(trimethoxysilyl)propylammonium chloride, tetradecyldimethyl(3-trimethoxysilylpropyl)ammonium chloride, and methyl(γ-methyldimethoxysilylpropyl)morpholinium chloride. One kind of them may be used alone, or two or more kinds thereof may be used in combination.

Examples of the cationic polymer include polylysine, polyallylamine, polyethyleneimine, polyarginine, polyhistidine, and chitosan. Among them, the cationic polymer is preferably polylysine. The polylysine is, for example, a polyamino acid in which an amino group at the ε-position of L-lysine is linked to a carboxy group via a peptide bond in a straight chain. Such polylysine contains the cationic group at the α-position at a high density, and therefore, the magnetic beads 2 having particularly high nucleic acid adsorption efficiency can be achieved.

The form of the cationic group is not particularly limited, and may be present in the compound in a state of, for example, an amidino group, an imidino group, a hydrazino group, or a pyridyl group.

The cationic group may be a group derived from a compound other than those described above. Examples of the compound other than those described above include a biological molecule containing an amino group, such as spermine and spermidine, a silane coupling agent containing a cationic group, and a thiol compound containing a cationic group.

### 2.2.3. Third Layer

The third layer 246 is provided between the first layer 242 and the second layer 244, and preferably includes a structure generated by a ring-opening reaction of an epoxy group. The third layer 246 is provided to contribute to enhancing the adhesion between the first layer 242 and the second layer 244. That is, the third layer 246 can favorably form the second layer 244 covering the first layer 242 with a high coverage ratio through the ring-opening reaction of the epoxy group. Accordingly, the magnetic beads 2 containing the cationic group at a higher density can be obtained.

As described below, the above structure can be formed, for example, by bringing an epoxy group into contact with an amino group, a hydroxyl group, a thiol group, or the like contained in a substance containing a cationic group to cause a ring-opening reaction in the epoxy group.

A substance containing an epoxy group is used to form the third layer 246. Examples of the substance containing an epoxy group include an epoxy group-containing coupling agent and a molecule containing two or more epoxy groups. The epoxy group-containing coupling agent contains an epoxy group as an organic functional group, and contains an alkoxy group, a halogen atom, or the like as a hydrolyzable group. Therefore, the epoxy group-containing coupling agent can efficiently introduce the epoxy group by reacting with a hydrolysate of the alkoxy group and bonding to a surface of the first layer 242.

Examples of the epoxy group-containing coupling agent include 3-glycidyloxypropyltrimethoxysilane, 3-glycidyloxypropyltriethoxysilane, 3-glycidyloxypropyl(dimethoxy)methylsilane, and 2-(3,4-epoxycyclohexyl)ethyltrimethoxysilane. One kind or two or more kinds of them may be used.

The third layer 246 may be provided as necessary, and for example, when the second layer 244 can be directly bonded to the first layer 242, the third layer 246 may be omitted. Examples of such a case include a case where the second layer 244 is formed using a coupling agent containing a cationic group, a thiol compound containing a cationic group, or the like. The former is bonded to the first layer 242 through a coupling reaction. The latter is bonded to the first layer 242 via an Au—S bond.

### 2.3. Characteristics of Magnetic Beads

An average particle diameter D50 of the magnetic beads 2 is preferably 0.5 μm or more and 30 μm or less, more preferably 1 μm or more and 20 μm or less, still more preferably 2 μm or more and 15 μm or less, and particularly preferably 3 μm or more and 10 μm or less. When the average particle diameter D50 of the magnetic beads 2 is within the above-described range, a specific surface area of the magnetic beads 2 can be sufficiently large, and an attractive force and an adsorption force suitable for magnetic separation can be generated in the magnetic beads 2. In addition, aggregation of the magnetic beads 2 can be prevented, and dispersibility can be improved.

When the average particle diameter D50 of the magnetic beads 2 is less than the above-described lower limit, the value of the magnetization of the magnetic beads 2 is reduced, and the magnetic beads 2 are likely to aggregate. As a result, there is a concern that the adsorption efficiency of the nucleic acid is reduced, or the contaminants are likely to be mixed. In addition, the movement speed of the magnetic bead 2 may decrease, and the time required for magnetic separation may increase. On the other hand, when the average particle diameter D50 of the magnetic beads 2 exceeds the upper limit, the specific surface area of the magnetic beads 2 is reduced, and therefore, a sufficient amount of the nucleic acid cannot be adsorbed, and a recovery rate (%) of the nucleic acid may decrease. In addition, the magnetic bead 2 is likely to settle, the magnetic bead 2 that can contribute to the extraction of the nucleic acid may decrease, and the recovery rate (%) of the nucleic acid may decrease.

The average particle diameter D50 of the magnetic bead 2 can be obtained from a cumulative distribution curve obtained from a volume-based particle size distribution measured by a laser diffraction and dispersion method. Specifically, in the cumulative distribution curve, a particle diameter (median diameter) where a cumulative value is 50% from a small diameter side is the average particle diameter D50 of the magnetic bead 2. Examples of a device that measures the particle size distribution by using the laser diffraction/scattering method include MT3300 series manufactured by MicrotracBEL Corp.

A saturation magnetization of the magnetic metal particle 22 is preferably 50 emu/g or more, more preferably 80 emu/g or more, and still more preferably 100 emu/g or more. The saturation magnetization is a magnetization value in a case where a magnetization exhibited by a magnetic material when a sufficiently large magnetic field is externally applied is constant regardless of the magnetic field. When the saturation magnetization is within the above range, a function as a magnetic material can be sufficiently exhibited. Specifically, since a movement speed of the magnetic bead 2 in a magnetic field can be increased, a time required for magnetic separation can be shortened. The saturation magnetization of the magnetic metal particle 22 affects an adsorption force when being fixed by an external magnetic field. When the saturation magnetization is within the above range, a sufficiently high adsorption force can be obtained, and therefore, when the liquid 3 is discharged in a state in which the magnetic bead 2 is immobilized, the magnetic bead 2 can be prevented from being discharged together with the liquid 3. Accordingly, a decrease in nucleic acid recovery rate (%) due to a decrease in the magnetic beads 2 can be prevented.

An upper limit value of the saturation magnetization of the magnetic metal particle 22 is not particularly limited, and is preferably 220 emu/g or less from the viewpoint of ease of selection of a material suitable for a balance between performance and cost.

The saturation magnetization of the magnetic metal particle 22 can be measured by a vibrating sample magnetometer (VSM) or the like. As the vibrating sample magnetometer, for example, TM-VSM1230-MHHL manufactured by Tamagawa Seisakusyo Co., Ltd. may be used. A maximum applied magnetic field when measuring the saturation magnetization is, for example, 0.5 T or more.

A coercive force Hc of the magnetic metal particle 22 is preferably 100 A/m or less, more preferably 80 A/m or less, still more preferably 60 A/m or less, and particularly preferably 50 A/m or less. The coercive force Hc refers to a value of an external magnetic field in an opposite direction required to return a magnetized magnetic material to an unmagnetized state. That is, the coercive force Hc means a resistance force against an external magnetic field. As the coercive force Hc of the magnetic metal particle 22 decreases, the magnetic beads 2 are less likely to aggregate even when being switched from a state in which a magnetic field is applied to a state in which no magnetic field is applied, and the magnetic beads 2 can be uniformly dispersed in the liquid 3. Further, even when the switching of the magnetic field application is repeated, the redispersibility of the magnetic beads 2 can be improved. A lower limit of the coercive force Hc of the magnetic metal particle 22 is not particularly limited, and is preferably 5 A/m or more from the viewpoint of ease of selection of a material suitable for a balance between performance and cost.

The coercive force Hc of the magnetic metal particle 22 can be measured by a vibrating sample magnetometer or the like in the same manner as the saturation magnetization described above. A maximum applied magnetic field when measuring the coercive force Hc is, for example, 15 kOe.

### 2.4. Method for Producing Magnetic Beads

Next, an example of a method of producing the magnetic beads 2 will be described. In the following description, the method for producing the magnetic bead 2 illustrated in FIG. 6 will be described as an example.

First, the magnetic metal particles 22 are prepared. The magnetic metal particles 22 are produced using a method according to a general metal powder production method. Examples of the production method include a melting process in which a metal is melted, solidified, and powdered, a chemical process in which a powder is produced by a reduction method or a carbonyl method, and a mechanical process in which a metal having a larger shape such as an ingot is mechanically pulverized to obtain a powder. Among them, a melting process such as an atomization method is suitable for producing the magnetic metal particles 22.

Next, the first layer 242 is formed on the surface of the magnetic metal particle 22. Examples of a method for forming the first layer 242 include a wet formation method such as a sol-gel method and a plating method and a dry formation method such as a vapor-phase deposition method. Among them, a Stöber method, which is a type of the sol-gel method, or an atomic layer deposition (ALD) method may be preferably used.

Next, an epoxy group is formed on the first layer 242. The epoxy group is introduced, for example, as follows.

First, a solution containing a substance containing an epoxy group is brought into contact with the surface of the first layer 242. At this time, a catalyst may be added to the solution together with the solvent. Examples of the catalyst include N-ethyldiisopropylamine. Next, the substance in contact with the surface of the first layer 242 reacts with the surface of the first layer 242. The reaction conditions at this time are known conditions applied to the coupling reaction and the like described above. Accordingly, an epoxy group is introduced onto the first layer 242. The substance containing an epoxy group is a precursor of the third layer 246.

Next, the second layer 244 is formed on the precursor of the third layer 246. The second layer 244 is formed, for example, as follows.

First, a solution containing a substance containing a cationic group is brought into contact with the precursor of the third layer 246. Next, the substance containing a cationic group reacts with the precursor of the third layer 246. The reaction conditions at this time are known conditions applied to the ring-opening reaction of the epoxy group described above. Accordingly, the third layer 246 including the structure generated by the ring-opening reaction of the epoxy group is formed. In addition, among the substances containing a cationic group, some amino groups, hydroxyl groups, thiol groups, or the like react with epoxy groups to be bonded thereto. Accordingly, the second layer 244 containing a cationic group is formed on the third layer 246.

As described above, the magnetic bead 2 including the magnetic metal particle 22 and the coating layer 24 having a multilayer structure covering the surface thereof can be obtained.

### 3. Biological Material Purification Kit

Next, a biological material purification kit according to the embodiment will be described.

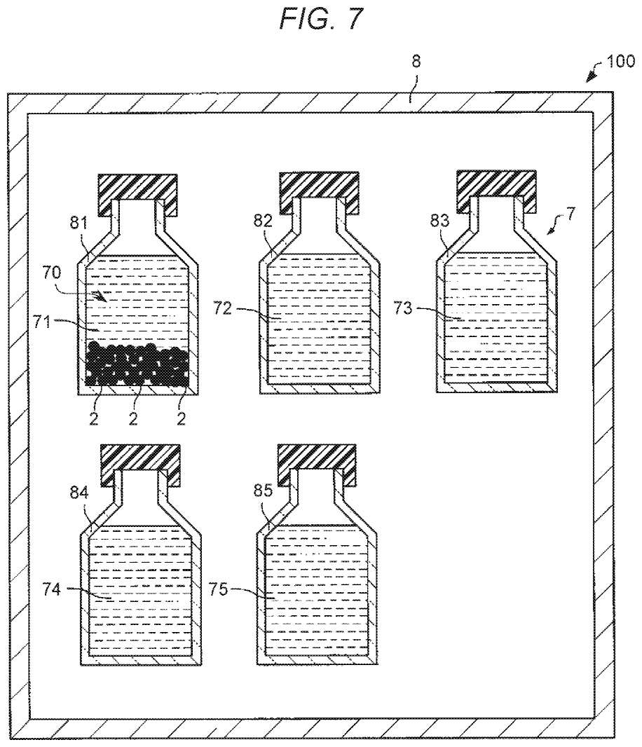

FIG. 7 is a cross-sectional view schematically illustrating a biological material purification kit 100 according to the embodiment.

The biological material purification kit 100 illustrated in FIG. 7 includes a biological material purification reagent set 7 and a housing 8 housing the biological material purification reagent set 7.

The biological material purification reagent set 7 includes a magnetic bead dispersion liquid 70 (magnetic bead dispersion liquid according to the embodiment), a nucleic acid adsorption reagent 72, a first cleaning reagent 73, a second cleaning reagent 74, and a nucleic acid desorption reagent 75.

According to such a configuration, the biological material purification kit 100 useful for the biological material purification method described above can be obtained.

The magnetic bead dispersion liquid 70 is accommodated in a reagent container 81. The magnetic bead dispersion liquid 70 contains the magnetic beads 2 and a dispersion medium 71 in which the magnetic beads 2 are dispersed. Examples of the dispersion medium 71 include water such as sterile water. Various additives may be added to the dispersion medium 71. Examples of the additive include the salts, buffers, and organic solvents described above.

The nucleic acid adsorption reagent 72 is accommodated in a reagent container 82. The nucleic acid adsorption reagent 72 contains, for example, a nucleic acid adsorption liquid and an additive.

The first cleaning reagent 73 is accommodated in a reagent container 83. The first cleaning reagent 73 contains, for example, a first cleaning liquid and an additive.

The second cleaning reagent 74 is accommodated in a reagent container 84. The second cleaning reagent 74 contains, for example, a second cleaning liquid and an additive. The components of the second cleaning liquid are different from those of the first cleaning liquid.

The nucleic acid desorption reagent 75 is accommodated in a reagent container 85. The nucleic acid desorption reagent 75 contains, for example, a nucleic acid desorption liquid and an additive.

The housing 8 has, for example, a bottomed box shape. The shape and material of the housing 8 are not particularly limited as long as the biological material purification reagent set 7 can be accommodated therein.

The biological material purification kit 100 may contain at least the magnetic bead dispersion liquid 70, and at least one of the nucleic acid adsorption reagent 72, the first cleaning reagent 73, the second cleaning reagent 74, the nucleic acid desorption reagent 75, and the housing 8 may be omitted.

### 4. Effects of Embodiment

As described above, the biological material purification method according to the embodiment includes the adsorption step S202, the separation step S204, and the desorption step S208. In the adsorption step S202, the biological material is adsorbed to the magnetic beads 2 in the mixed liquid obtained by mixing the magnetic beads 2 and the biological material. In the separation step S204, the magnetic beads 2 to which the biological material is adsorbed are separated from the mixed liquid. In the desorption step S208, the biological material is desorbed from the magnetic beads 2.

The magnetic bead 2 includes the magnetic metal particle 22 and the coating layer 24 that covers the surface of the magnetic metal particle 22 and contains a cationic group.

When the pH of the nucleic acid adsorption liquid (the liquid 3 in which the magnetic beads 2 are present) in the adsorption step S202 is denoted by pH(a), the pH of the nucleic acid desorption liquid (the liquid 3 in which the magnetic beads 2 are present) in the desorption step S208 is denoted by pH(d), and the salt concentration of the nucleic acid adsorption liquid (the liquid 3 in which the magnetic beads 2 are present) in the adsorption step S202 is denoted by C(a), the following formulas (1), (2), and (3) are satisfied.

$$pH ( a ) \leq 7. \leq pH ( d ) \qquad ( 1 )$$

$$2. \leq pH ( d ) - pH ( a ) \qquad ( 2 )$$

$$C ( a ) \leq 2. [ M ] \qquad ( 3 )$$

According to such a configuration, the nucleic acid can be efficiently adsorbed to the magnetic beads 2 by the ionic interaction between the magnetic beads 2 containing the cationic group and the nucleic acid. In addition, adsorption of contaminants can be prevented while efficiently adsorbing and desorbing nucleic acids. As a result, it is possible to provide a biological material purification method capable of purifying a high-purity biological material in a high nucleic acid recovery rate (%).

In the biological material purification method according to the embodiment, when the temperature of the nucleic acid adsorption liquid (the liquid 3 in which the magnetic beads 2 are present) in the adsorption step S202 is denoted by T(a), and the temperature of the nucleic acid desorption liquid (the liquid 3 in which the magnetic beads 2 are present) in the desorption step S208 is denoted by T(d), the following formulas (4), (5), and (6) are preferably satisfied.

$$T ( a ) \leq T ( d ) \qquad ( 4 )$$

$$T ( a ) \leq 35 [ ° C . ] \qquad ( 5 )$$

$$20 [ ° C . ] \leq T ( d ) \qquad ( 6 )$$

According to such a configuration, the adsorption efficiency of the nucleic acid in the adsorption step S202 and the desorption efficiency of the nucleic acid in the desorption step S208 are increased.

In the biological material purification method according to the embodiment, the biological material may be a nucleic acid.

According to such a configuration, the nucleic acid can be efficiently purified.

In the biological material purification method according to the embodiment, the zeta potential of the magnetic beads 2 in the adsorption step S202 is preferably a positive zeta potential of 20 [mV] or more.

According to such a configuration, the magnetic beads 2 in the adsorption step S202 have sufficiently strong cationic properties. Therefore, in the magnetic beads 2 in the adsorption step S202, a stronger attractive force can be applied to the nucleic acid by the ionic interaction. Accordingly, the adsorption efficiency of the nucleic acid can be further increased.

The magnetic bead 2 according to the embodiment is a magnetic bead used for the biological material purification method according to the embodiment, and includes the magnetic metal particle 22 and the coating layer 24 that covers the surface of the magnetic metal particle 22. The coating layer 24 includes the first layer 242 containing at least one of an inorganic oxide and gold, and the second layer 244 provided at the first layer 242 and containing a cationic group.

According to such a configuration, in the biological material purification method according to the embodiment, the magnetic bead 2 can be efficiently adsorbed and efficiently desorbed through the ionic interaction between the magnetic bead 2 and the nucleic acid. As a result, the magnetic beads 2 contributing to the biological material purification method capable of purifying a high-purity biological material in a high nucleic acid recovery rate (%) can be obtained.

In the magnetic bead 2 according to the embodiment, the coating layer 24 may further include the third layer 246 provided between the first layer 242 and the second layer 244. In this case, the third layer 246 includes a structure generated by a ring-opening reaction of an epoxy group.

According to such a configuration, the third layer 246 contributes to enhancing the adhesion between the first layer 242 and the second layer 244. Accordingly, the magnetic beads 2 containing the cationic group at a higher density can be obtained.

In the magnetic bead 2 according to the embodiment, the magnetic metal particle 22 may contain an Fe-based amorphous alloy.

According to such a configuration, the magnetic permeability of the magnetic metal particle 22 is high, and the coercive force is low, and therefore, it is possible to achieve the magnetic bead 2 having excellent separability in magnetic separation and excellent re-dispersibility.

The magnetic bead dispersion liquid 70 according to the embodiment includes the magnetic beads 2 (magnetic beads according to the embodiment) and the dispersion medium 71 that disperses the magnetic beads 2.

According to such a configuration, the magnetic bead dispersion liquid 70 useful for the biological material purification method described above can be obtained.

The biological material purification kit 100 according to the embodiment includes the magnetic bead dispersion liquid 70 (magnetic bead dispersion liquid according to the embodiment).

According to such a configuration, the biological material purification kit 100 useful for the biological material purification method described above can be obtained.

Although the biological material purification method, the magnetic bead, the magnetic bead dispersion liquid, and the biological material purification kit according to the present disclosure have been described above based on the illustrated embodiments, the present disclosure is not limited thereto. For example, the biological material purification method according to the present disclosure may be a method in which a step for any purpose is added to the embodiment. In each of the magnetic bead, the magnetic bead dispersion liquid, and the biological material purification kit according to the present disclosure, any configuration may be added to the embodiment, or a part of the configuration of the embodiment may be replaced with another configuration having the same function.

### Examples

Next, specific examples of the present disclosure will be described.

### 5. Production of Magnetic Beads and Nucleic Acid Purification

Magnetic beads in Examples and Comparative Examples were produced as follows, and then, nucleic acid purification was performed using the produced magnetic beads.

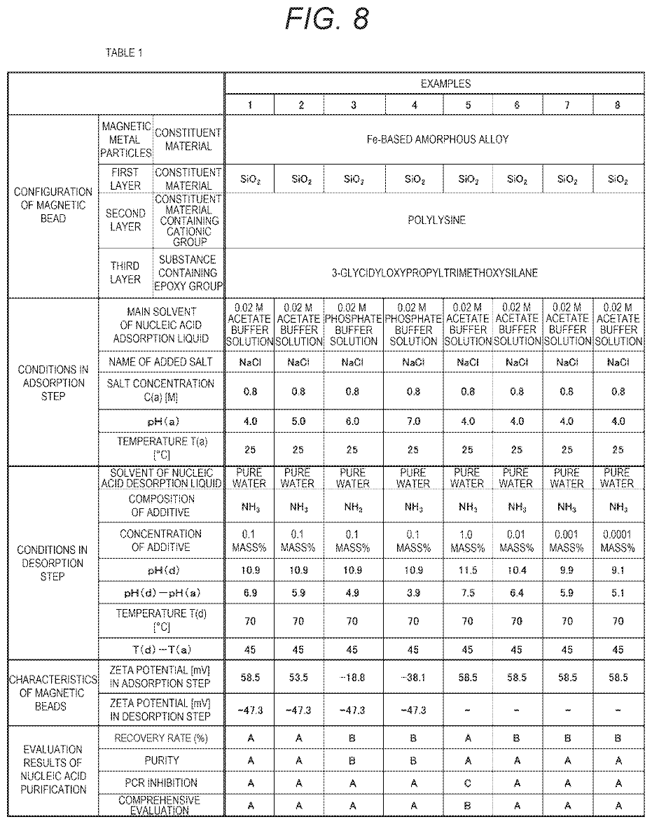

FIG. 8 is Table 1 illustrating conditions for the nucleic acid purification using magnetic beads in Examples and Comparative Examples, and evaluation results of the nucleic acid purification.

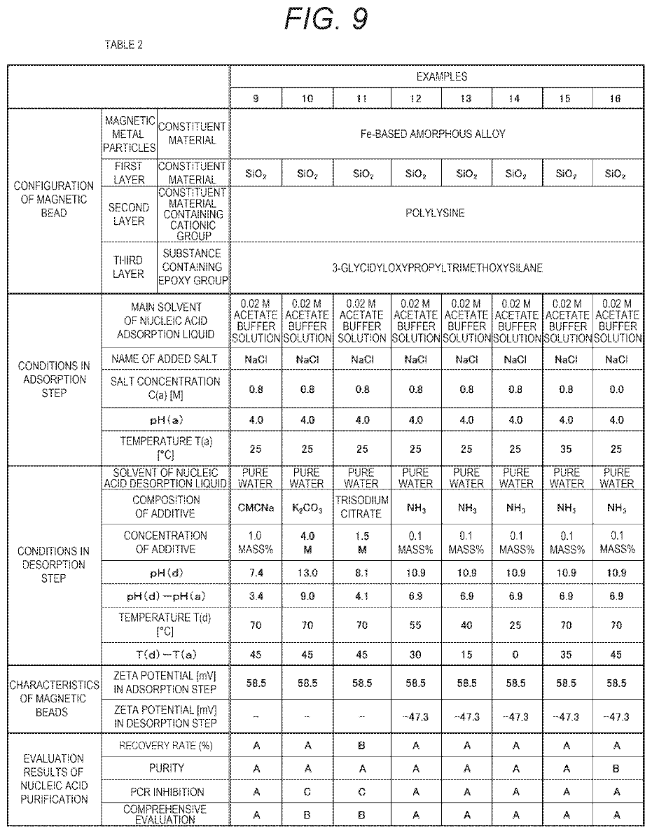

FIG. 9 is Table 2 illustrating conditions for nucleic acid purification using the magnetic beads in Examples and evaluation results of nucleic acid purification.

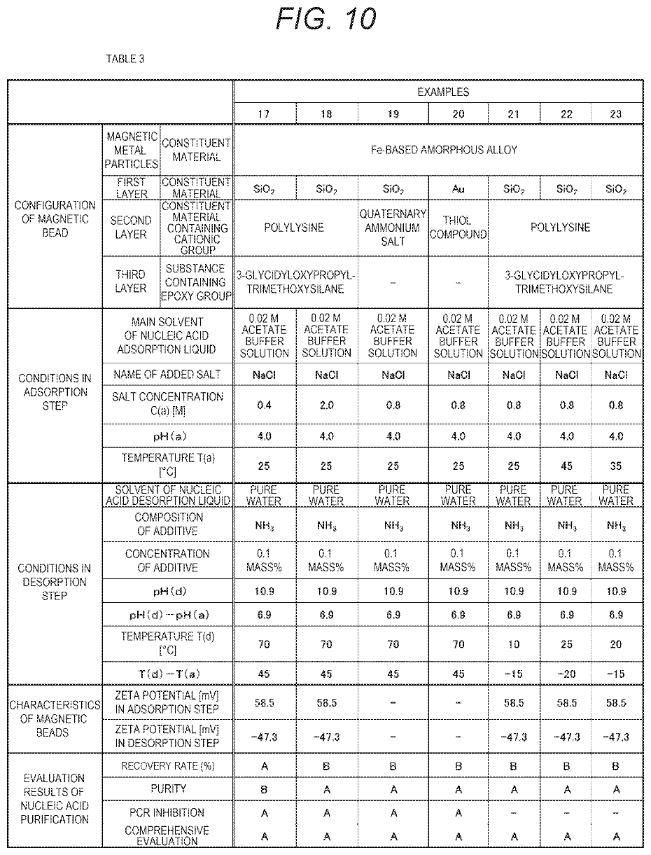

FIG. 10 is Table 3 illustrating conditions for nucleic acid purification using the magnetic beads in Examples and evaluation results of nucleic acid purification.

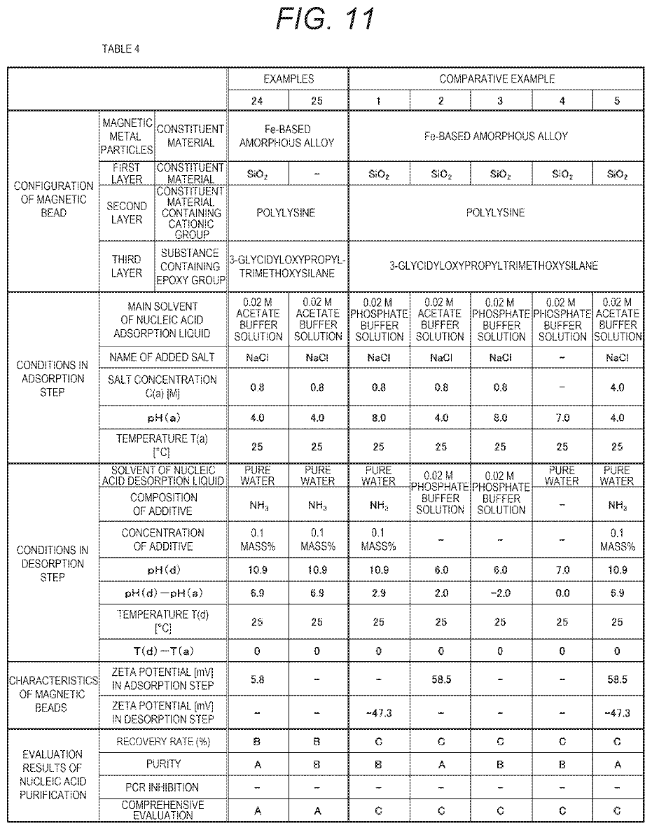

FIG. 11 is Table 4 illustrating conditions for the nucleic acid purification using magnetic beads in Examples and Comparative Examples, and evaluation results of the nucleic acid purification.

### 5.1. Example 1

First, the production of magnetic beads and nucleic acid purification of Example 1 will be described.

### 5.1.1. Production of Magnetic Bead

First, an Fe-based amorphous alloy powder produced according to a water atomization method was prepared as magnetic metal particles.

Next, tetraethoxysilane (TEOS) was brought into contact with the surface of the magnetic metal particles according to a Stöber method, and then firing was performed to form a first layer made of SiO₂.

Next, 1.0 g of the magnetic metal particles having the first layer formed thereon was weighed into a glass bottle.

Next, 4.6 g of ethanol containing catalyst N-ethyldiisopropylamine in a ratio of 0.25 mass % and containing pure water in a ratio of 5.0 mass % was added to the glass bottle.

Next, 0.13 g of 3-glycidyloxypropyltrimethoxysilane (a substance containing an epoxy group) was added to the glass bottle. Then, tumble stirring was performed for 3.5 hours in an environment at 37° C. Accordingly, an epoxy group was introduced onto the first layer. The magnetic metal particles into which the epoxy group has been introduced are referred to as epoxy group-modified particles.

Next, 100 μL of a suspension liquid containing 240 mg of epoxy group-modified particles was weighed into an microcentrifuge tube.

Next, the solvent of the suspension liquid in the microcentrifuge tube was replaced with a 0.1 M phosphate buffer solution adjusted to pH 7.4.

Next, 100 μL of a solution containing polylysine was added to the microcentrifuge tube, followed by stirring with a vortex mixer for 2 minutes.

Next, 100 μL of a 3.0 M ammonium sulfate solution was added to the microcentrifuge tube. Then, tumble stirring was performed for 48 hours in an environment at 23° C. Accordingly, the epoxy group-modified particles and polylysine react with each other to introduce a cationic group (amino group). As a result, a coating layer in which the first layer, the third layer, and the second layer were laminated in this order was formed on the surface of the magnetic metal particles to obtain magnetic beads.

### 5.1.2. Nucleic Acid Purification

First, a magnetic bead dispersion liquid containing 39.2 mg of the magnetic beads of Example 1 was divided into microcentrifuge tubes.

Next, an adsorption step was performed. Specifically, a dispersion medium of the magnetic bead dispersion liquid in each of the microcentrifuge tubes was replaced with a nucleic acid adsorption liquid. Then, the magnetic beads and the nucleic acid adsorption liquid were combined and adjusted to 715 μL. Subsequently, 200 μL of an artificial serum containing 0.2 μg, 1.0 μg, or 2.0 μg of a nucleic acid was charged into each of the microcentrifuge tubes, followed by performing tumble stirring at room temperature.

Next, a separation step was performed. Specifically, a magnetic separation treatment and a liquid discharging treatment were performed on the substances accommodated in the microcentrifuge tube.

Next, a washing step was performed. Specifically, the substances accommodated in the microcentrifuge tube were cleaned twice. Each cleaning is performed by adding 900 μL of a cleaning liquid and then performing a magnetic separation treatment and a liquid discharging treatment. A 0.02 M acetate buffer solution adjusted to pH 4.0 was used as the cleaning liquid.

Next, a desorption step was performed. Specifically, 50 μL of the nucleic acid desorption liquid was added to the microcentrifuge tube, and vibration stirring was performed by a block bath shaker at 1500 rpm for 10 minutes. An ammonia (NH₃) solution having a concentration illustrated in Table 1 was added as an additive to the microcentrifuge tube. The temperature of the microcentrifuge tube during stirring was 25° C., 40° C., 55° C., or 70° C. Next, the magnetic separation treatment and the liquid discharging treatment were performed, and the nucleic acid desorption liquid in the supernatant was collected.

Table 1 (FIG. 8) illustrates the conditions in the adsorption step and the desorption step, and the characteristics of the magnetic beads used.

Other characteristics of the magnetic beads used are as follows.
Average particle diameter D50: 3.4 μm
Saturated magnetization: 110 emu/g
Coercive force: 50 A/m
Thickness of first layer containing SiO₂: 30 nm

### 5.2. Examples 2 to 25 and Comparative Examples 1 to 5

Magnetic beads were produced and nucleic acid purification was performed in the same manner as in Example 1 except that the configuration of the magnetic beads and the conditions of the nucleic acid purification were changed as illustrated in Table 1 (FIG. 8), Table 2 (FIG. 9), Table 3 (FIG. 10), or Table 4 (FIG. 11), respectively.

The “thiol compound” illustrated in Table 3 is a compound represented by a structural formula HS—(CH₂)₁₁—NMe₃⁺Cl⁻. Me represents a methyl group.

The “quaternary ammonium salt” illustrated in Table 3 is trimethyl[3-(trimethoxysilyl)propyl]ammonium chloride.

The thickness of the first layer containing Au was 5 nm.

In some Examples and Comparative Examples, the evaluation of the characteristics of the magnetic beads was omitted.

### 6. Evaluation of Nucleic Acid Purification

Next, the results of the nucleic acid purification in Examples and Comparative Examples were evaluated.

### 6.1. Evaluation of recovery rate (%) of Nucleic Acid

First, 2 μL of the collected nucleic acid desorption liquid was subjected to a 10 μL real-time PCR reaction system to acquire a Ct value. The Ct value is a Cycle threshold value, and is a numerical value indicating how many times amplification was performed until a target material reached a detectable threshold. More specifically, the Ct value is the number of cycles when an amplification product reaches a predetermined amount in PCR and a fluorescence luminance reaches a predetermined value (when a PCR amplification curve rises). Therefore, the smaller the Ct value, the higher the purification efficiency of the target material, and the shorter the test time.

Using a calibration curve illustrating a correlation between the Ct value and the nucleic acid concentration separately acquired using real-time PCR, the nucleic acid concentration of the nucleic acid desorption liquid was determined from the acquired Ct value. Then, the nucleic acid yield was calculated using the determined nucleic acid concentration and the amount of the nucleic acid desorption liquid. For all of the nucleic acid desorption liquids including the case where the Ct value was not obtained due to amplification inhibition or the like in real-time PCR, the nucleic acid concentration was determined using Qubit or Nanodrop, and the nucleic acid yield was calculated.

Next, the nucleic acid recovery rate (%) was calculated using the calculated nucleic acid yield and the input nucleic acid weight.

Next, the calculated nucleic acid recovery rate (%) was evaluated in view of the following evaluation criteria. Evaluation results are illustrated in Tables 1 to 4.
A: the nucleic acid recovery rate (%) is 80% or more
B: the nucleic acid recovery rate (%) is 50% or more and less than 80%
C: the nucleic acid recovery rate (%) is less than 50%

### 6.2. Evaluation of Purity of Nucleic Acid

First, a mass of a protein as a contaminant mixed into the collected nucleic acid desorption liquid was determined.

Next, a ratio of the mass of the protein mixed into the nucleic acid desorption liquid to a mass of the input protein was calculated as a contaminant mixing ratio.

Next, the purity of the nucleic acid was evaluated by comparing the calculated contaminant mixing ratio with the following evaluation criteria. Evaluation results are illustrated in Tables 1 to 4.
A: the contaminant mixing ratio is less than 0.1%
B: the contaminant mixing ratio is 0.1% or more and less than 0.2%
C: the contaminant mixing ratio is 0.2% or more

### 6.3. Evaluation of PCR Inhibition in Nucleic Acid Purification

First, in order to investigate the influence of nucleic acid desorption conditions (pH, additive material, temperature) on PCR inhibition, a nucleic acid solution obtained by adding nucleic acids to pure water so as to have a concentration of 0.25 [ng/uL] was prepared and used as a reference solution. Further, a nucleic acid solution obtained by adding nucleic acids to each nucleic acid desorption liquid so as to have a concentration of 0.25 [ng/uL] was prepared, and the same temperature as that applied in each desorption step was applied for 10 minutes. Thereafter, 2 μL of each solution was subjected to a 10 μL real-time PCR reaction system to obtain a Ct value.

Next, the obtained Ct value was compared with the following evaluation criteria to evaluate PCR inhibition in nucleic acid purification. Evaluation results are illustrated in Tables 1 to 4. In some Examples and Comparative Examples, the evaluation of PCR inhibition was omitted.
A: the PCR amplification curve rises within 40 cycles, and the Ct value is in the range of ±0.5 as compared with the reference solution
C: the PCR amplification curve does not rise within 40 cycles, or the PCR amplification curve rises within 40 cycles, but the Ct value exceeds ±0.5 compared with the reference solution

### 6.4. Comprehensive Evaluation

The comprehensive evaluation of the nucleic acid purification was performed by comparing the results of the above evaluations with the following evaluation criteria. Evaluation results are illustrated in Tables 1 to 4.
A: there is no evaluation value C in all the evaluation results
B: there is no evaluation value C in both the recovery rate (%) and the purity, and there is an evaluation value C in the PCR inhibition
C: there is an evaluation value C is at least one of the recovery rate (%) and the purity

From the evaluation results illustrated in Tables 1 to 4, the following can be recognized.
In the nucleic acid purification of each example, a high recovery rate (%) and high purity nucleic acid purification was able to be performed by using magnetic beads containing a cationic group and optimizing the pH and salt concentration of the liquid in each step.
The above results are considered to be obtained because selective adsorption of the nucleic acid in the adsorption step and efficient desorption of the nucleic acid in the desorption step was able to be achieved.
In the nucleic acid purification of each example, a high recovery rate (%) and high purity nucleic acid purification was able to be performed without using an adsorption promoting substance such as a chaotropic substance.
In the nucleic acid purification of some examples, the occurrence of PCR inhibition due to the conditions in each step was able to be prevented.

When the nucleic acid desorption liquid is not subjected to PCR as an application of a nucleic acid after nucleic acid purification, the occurrence of PCR inhibition may not be a problem. In addition, even under the condition that PCR inhibition occurs, the nucleic acid after nucleic acid purification can be used as a high recovery rate (%) and high purity nucleic acid depending on the application of the nucleic acid desorption liquid.

What is claimed is:

1. A biological material purification method comprising:
an adsorption step of adsorbing, in a mixed liquid obtained by mixing a magnetic bead and a biological material, the biological material to the magnetic bead;
a separation step of separating, from the mixed liquid, the magnetic bead to which the biological material is adsorbed; and
a desorption step of desorbing the biological material from the magnetic bead, wherein
the magnetic bead includes a magnetic metal particle, and a coating layer covering a surface of the magnetic metal particle and containing a cationic group, and
the following formulas (1), (2), and (3) are satisfied:
$$pH ( a ) \leq 7. \leq pH ( d ) \qquad ( 1 )$$
$$2. \leq pH ( d ) - pH ( a ) \qquad ( 2 )$$
$$C ( a ) \leq 2. [ M ] , \qquad ( 3 )$$
where pH(a) represents a pH of a liquid in which the magnetic bead is present in the adsorption step, pH(d) represents a pH of a liquid in which the magnetic bead is present in the desorption step, and C(a) represents a salt concentration of the liquid in which the magnetic bead is present in the adsorption step.

2. The biological material purification method according to claim 1, wherein
the following formulas (4), (5), and (6) are satisfied:
$$T ( a ) \leq T ( d ) \qquad ( 4 )$$
$$T ( a ) \leq 35 [ ° C . ] \qquad ( 5 )$$
$$20 [ ° C . ] \leq T ( d ) \qquad ( 6 )$$
where T(a) represents a temperature of the liquid in which the magnetic bead is present in the adsorption step, and T(d) represents a temperature of the liquid in which the magnetic bead is present in the desorption step.

3. The biological material purification method according to claim 1, wherein
the biological material is a nucleic acid.

4. The biological material purification method according to claim 3, wherein
a zeta potential of the magnetic bead in the adsorption step is a positive zeta potential of 20 [mV] or more.

5. A magnetic bead, which is the magnetic bead for use in the biological material purification method according to claim 1, the magnetic bead comprising:
the magnetic metal particle; and
the coating layer configured to cover the surface of the magnetic metal particle, wherein
the coating layer includes a first layer containing at least one of an inorganic oxide and gold, and a second layer provided at the first layer and containing the cationic group.

6. The magnetic bead according to claim 5, wherein
the coating layer further includes a third layer provided between the first layer and the second layer, and
the third layer includes a structure generated by a ring-opening reaction of an epoxy group.

7. The magnetic bead according to claim 5, wherein
the magnetic metal particle contains an Fe-based amorphous alloy.

8. A magnetic bead dispersion liquid comprising:
the magnetic bead according to claim 5; and
a dispersion medium in which the magnetic bead is dispersed.

9. A biological material purification kit comprising:
the magnetic bead dispersion liquid according to claim 8.
